(19) United States
(12) Patent Application Publication
(10) Pub. No.: US 2019/0350622 A1
(43) Pub. Date: Nov. 21, 2019

(54) Colpotomy System with Uterine Manipulator and Electrocautery for Total Laparoscopic Hysterectomy

(71) Applicant: Tarek Ahmed Nabil Abou El Kheir, Salt Lake City, UT (US)

(72) Inventor: Tarek Ahmed Nabil Abou El Kheir, Salt Lake City, UT (US)

(21) Appl. No.: 15/985,650

(22) Filed: May 21, 2018

(51) Int. Cl. A61B 17/42; A61B 17/32; A61B 18/14; A61B 34/00

(52) U.S. Cl. CPC A61B 17/4241; A61B 17/320016; A61B 2017/00743; A61B 34/70; A61B 18/14

(57) ABSTRACT

A device for facilitating a total laparoscopic hysterectomy includes a cervical cup adapted to abut the cervicovaginal junction. An electrocautery wire has an uninsulated portion housed in the cervical cup which includes a channel for housing and controlled releasing the uninsulated portion. An introducer is coupled to the electrocautery wire and configured to penetrate the cervicovaginal junction thereby introducing the electrocautery wire into the intra-abdominal cavity. A shaft secures the electrocautery wire and pulling the electrocautery wire causes a controlled release of the uninsulated portion along a predetermined circumferential path around the cervix along the channel. Thus, the vagina is severed from the cervix along the circumference of the cervicovaginal junction.

### BACKGROUND

Total hysterectomy procedures involving the use of a colpotomy ring are known in the art, including the removal of the patient's uterus and cervix. In such a procedure, the patient's vagina is separated from the patient's uterus and cervix prior to extraction. Performing that portion of the procedure is difficult because there currently exists no way of uniformly cutting around the patient's cervicovaginal junction and vaginal fornix. Under current practices, several incisions must be made laparoscopically, which is time consuming and prone to error due to the difficulty in locating and manipulating the patient's cervicovaginal junction and vaginal fornices, adjacent the uterus.

Hence, what is needed is a device for severing a patient's vagina from the patient's uterus and cervix in a minimally invasive and controlled manner. There is also a need for such a device that is installed on an otherwise conventional colpotomy ring and uterine sound assembly. Finally there is a need for such a device that uses limited electrocautery to sever the patient's uterus from the vagina without impacting the surrounding tissue, and which may be subsequently removed through a laparoscopy port or through the vagina.

### SUMMARY

A device is provided for facilitating a total laparoscopic hysterectomy including removing a patient's uterus and cervix, when the uterus and a portion of the patient's cervix are located inside an intra-abdominal cavity and a vagina is circumferentially attached to the cervix creating a cervicovaginal junction, wherein the cervix has a cervical canal, the uterus has an intrauterine cavity, and the intra-abdominal cavity includes a laparoscopic access installed through the anterior abdominal wall.

Among other features, the device includes a cervical cup having a cervical cup proximal end and a cervical cup distal end. The cervical cup distal end is adapted for embracing the cervix with the cervical cup distal end abutting the cervicovaginal junction, and the cervical cup proximal end extending away from the cervix. An electrocautery wire having an electrocautery wire proximal end and an electrocautery wire distal end is included within the cervical cup, and the electrocautery wire distal end includes an uninsulated portion. The cervical cup distal end includes a channel for housing the uninsulated portion of the electrocautery wire, and the channel is configured for a controlled release of the uninsulated portion of the electrocautery wire distal end.

An introducer is provided, having an introducer proximal end and an introducer distal end, the introducer proximal end is releasably coupled to the electrocautery wire proximal end, and the introducer is configured to penetrate the cervicovaginal junction upon activation of the introducer distal end, thereby introducing the electrocautery wire proximal end into the intra-abdominal cavity. A shaft is provided, the shaft having a shaft proximal end, and a shaft distal end, wherein the shaft distal end is configured for introduction into the intra-abdominal cavity through the access installed in the anterior abdominal wall.

The shaft is configured to secure the electrocautery wire proximal end through the shaft distal end and move the electrocautery wire proximal end through the shaft toward the shaft proximal end, thereby pulling the electrocautery wire uninsulated portion from the channel and causing a controlled release of the uninsulated portion along a predetermined circumferential path around the cervix along the channel. Thus, the vagina is severed from the cervix along the circumference of the cervicovaginal junction.

The apparatus preferably also includes two sliders inside the channel, with each slider configured to slide in an opposing 180 degree semi-circular track until the two sliders meet. Each slider preferably includes a duct having an inlet and an outlet, and the uninsulated portion of the electrocautery wire passes through the duct of each slider from the inlet to the outlet, emerging from each outlet toward the outside of the channel. That arrangement allows the uninsulated portion to be released along the channel and for the electrocautery wire to disengage the cervical cup through the outlet of each duct.

The apparatus preferably also includes two flexible insulated sheaths, wherein each flexible insulated sheath has a sheath proximal end and a sheath distal end. Each sheath distal end is individually coupled to a slider, such that each flexible insulated sheath can move a predetermined distance toward or away from each slider. Each flexible insulated sheath also preferably includes a duct having a duct proximal end and a duct distal end. Each duct distal end faces each slider and each duct proximal end is releasably coupled to the shaft distal end, such that each of the ducts house a portion of the electrocautery wire, with the electrocautery wire extended from the duct distal end to the duct proximal end.

The flexible insulated sheaths are configured such that pulling on the proximal end of the shaft with the shaft distal end coupled to each flexible insulated sheath causes each of the flexible insulated sheaths to move away from each slider a predetermined distance, exposing portion of the uninsulated electrocautery wire. The electrocautery wire is also configured such that moving the proximal end through the long shaft causes the electrocautery wire to move through the duct in a manner that the uninsulated portion extends only between a slider and a flexible insulated sheath, thereby causing the uninsulated portion to slide through a predetermined circular path around the cervix.

The apparatus preferably also includes an elongated curved stem having a stem proximal end, a stem distal end and a first curved axis, the stem distal end is adapted to be introduced through the cervical canal into the intrauterine cavity, thereby providing a means for manipulating the uterus, and the proximal end includes a handle. The device preferably includes an elongated curved cylinder having a proximal end, a distal end, with a duct along its length and a second curved axis matching the first curved axis. The elongated curved cylinder is preferably shorter in length than the elongated curved stem, and the distal end of the elongated cylinder is adapted to be coupled with the proximal end of the cervical cup. This allows them to move as one unit, wherein the elongated curved stem is configured for sliding through the duct of the elongated curved cylinder and adapted to be locked in place at a predetermined distance set by the operator.

The device preferably also includes a cable having a cable proximal end and a cable distal end. The cable distal end is attached to the flexible insulated sheath proximal end and the cable proximal end is configured for releasably coupling to the shaft distal end, thereby facilitating temporary coupling of the shaft with the insulated flexible sheath, and allowing them to move as a single unit. The cable proximal end is also configured to be releasably coupled to the introducer proximal end alongside the electrocautery wire proximal end, so that actuating the introducer distal end pushes the introducer forward, penetrating a small portion of the cervicovaginal junction, thereby introducing both the cable proximal end and the electrocautery wire proximal end to the inside of the intra-abdominal cavity.

The semicircular tracks of the channel at the cervical cup distal end are preferably tilted interiorly, thereby causing the sliders to be tilted interiorly along the track. This causes the angle of the exposed portion of the electrocautery wire extending between each slider and each flexible insulated sheath to be tilted interiorly closer to the cervix, thereby causing the cutting area severed by the uninsulated portion in the cervicovaginal junction to be closer to the cervix and away from other body organs. Additionally, a portion of the shaft may be flexible, a portion of the shaft may be bifurcated, and the shaft may be insulated.

In various alternative embodiments, an optical/light system terminating at the cervical cup. The shaft includes sliding bars configured to move the electrocautery wire through the shaft. The uninsulated portion of the electrocautery wire distal end may be divided into two arms, wherein each arm is coupled to an opposing side of a non-conductive element, thereby allowing the electrocautery wire to function using a bipolar electrocautery technique. Additionally, the non-conductive element may be anchored in place in the channel at the cervical cup distal end. In various alternative embodiments, the device may also include a component selected from the group consisting of a monopolar wired electrocautery energy generator, a bipolar wired electrocautery energy generator, a monopolar batteries operated electrocautery energy generator, a bipolar batteries operated electrocautery energy generator, a pedal activating electrocautery, a manual activating electrocautery, and a button activating electrocautery, a gas seal vaginal element, a gas seal vaginal balloon occluder, and at least one handle.

The device may also be characterized as a device to facilitate a total laparoscopic hysterectomy of a uterus, wherein the uterus and a portion of a cervix are inside an intra-abdominal cavity and a vagina is circumferentially attached to the cervix creating a cervicovaginal junction, wherein the cervix has a cervical canal, the uterus has an intrauterine cavity, and the intra-abdominal cavity includes an access installed through the anterior abdominal wall. The device in this embodiment includes a cervical cup having a cervical cup distal end adapted to abut the cervicovaginal junction, and a cervical cup proximal end extending away from the cervix. An electrocautery wire is provided having an insulated portion, and an uninsulated portion disposed in the cervical cup distal end.

The cervical cup distal end preferably includes a channel housing the uninsulated portion, the channel comprising a pair of sliders configured to move through the channel and release of the uninsulated portion of the electrocautery wire. An introducer having an introducer proximal end and an introducer distal end is included, with the introducer proximal end releasably coupled to the electrocautery wire insulated portion, and the introducer slidable relative to the cervical cup, such that the proximal end is configured to penetrate the cervicovaginal junction upon activation of the introducer distal end, thereby introducing the electrocautery wire insulated portion into the intra-abdominal cavity.

The device preferably also includes a shaft having a shaft proximal end, and a shaft distal end, wherein the shaft distal end is configured for introduction into the intra-abdominal cavity through the access installed in the anterior abdominal wall. The shaft is further configured to secure the electrocautery wire insulated portion through the shaft distal end and urge the electrocautery wire through the shaft toward the shaft proximal end, thereby pulling the electrocautery wire uninsulated portion through the sliders and around the channel, and thereby cause a controlled release of the uninsulated portion along a predetermined circumferential path around the cervix.

A method is also provided for separating a patient's cervix from the patient's vagina at the cervicovaginal junction. The method includes the steps of providing a colpotomy cup assembly having a colpotomy ring, providing an electrocautery wire on the colpotomy cup assembly, passing the electrocautery wire through an insulated flexible sheath, providing a tensioner cable on the insulated flexible sheath.

An introducer trocar is provided on the colpotomy cup assembly in a sliding relationship to the colpotomy ring, and the introducer trocar is anchored to the electrocautery wire and the tensioner cable to the introducer trocar. The colpotomy ring is placed against the cervicovaginal junction, and urged through the cervicovaginal junction, thereby carrying the electrocautery wire and tensioner cable through the cervicovaginal junction. The electrocautery wire and the tensioner cable are captured with a laparoscopic shaft assembly, placed under tension, and the electrocautery wire is pulled through the insulated flexible sheath while causing the electrocautery wire to travel around the colpotomy ring.

In various alternative embodiments of the method, two sliders are provided and coupled to the two flexible insulated sheaths. The two sliders are configured to travel along with the flexible insulated sheaths around two semi-circular portions of the colpotomy ring and control the release of the electrocautery wire from the colpotomy ring. Additionally, a first handle may be provided for moving the tensioner cable into the laparoscopic shaft assembly, such that the flexible insulated sheath abuts the laparoscopic shaft assembly, a second handle may be provided on the laparoscopic shaft assembly for drawing the electrocautery wire through the flexible insulated sheath, and a release system may be provided comprising a colpotomy cup roller affixed to a roller rod. When the electrocautery wire is urged through the colpotomy cup roller, such that the colpotomy cup roller travels around the colpotomy ring, a channel provided in the colpotomy ring is configured to allow the colpotomy cup roller to pass therethrough. By providing a roller rod channel and a spring in the flexible insulated sheath, the roller rod channel and spring are configured to arrest movement of the insulated sheath away from the colpotomy ring.

### BRIEF DESCRIPTION OF THE FIGURES

FIG. 1 illustrates a perspective view of a uterine colpotomy system assembly;

FIG. 2a illustrates a perspective view of an accordion folded electrocautery wire and tensioner cable, including flexible insulated sheaths and a release system;

FIG. 2b illustrates a perspective view of the accordion folded electrocautery wire and tensioner cable attached to an introducer trocar, including flexible insulated sheaths and a release system;

FIG. 3 illustrates a perspective view of the uterine colpotomy system assembly, with the electrocautery wire and tensioner cable extending into a laparoscopy shaft assembly;

FIG. 4a illustrates a perspective view of the uterine colpotomy system assembly with the electrocautery wire, tensioner cable, and flexible insulated sheaths extended.

FIG. 4b illustrates a perspective view of a uterine sound;

FIG. 4c illustrates a perspective view of a colpotomy cup assembly;

FIG. 4d illustrates the terminal ends of the electrocautery wire and tensioner cable;

FIG. 5a illustrates a perspective view of the colpotomy cup assembly, including a colpotomy ring bearing the electrocautery wire and release system in a channel;

FIG. 5b illustrates a perspective view of the release system;

FIG. 5c illustrates a partial cut-away perspective view of the release system;

FIG. 6a illustrates an enlarged view of the colpotomy ring;

FIG. 6b illustrates an enlarged view of the electrocautery wire and release mechanism, including an electrocautery disc;

FIG. 7a1 illustrates a perspective view of a portion of the colpotomy ring and the release mechanism in a retracted configuration.

FIG. 7b1 illustrates a perspective view of a portion of the colpotomy ring and the release mechanism in a non-retracted configuration.

FIG. 7a2 illustrates a perspective view of the release mechanism in a retracted configuration.

FIG. 7b2 illustrates a perspective view of the release mechanism in a non-retracted configuration.

FIG. 7a3 illustrates a cut-away perspective view of the release mechanism in a retracted configuration.

FIG. 7b3 illustrates a cut-away perspective view of the release mechanism in a non-retracted configuration.

FIG. 8a illustrates a perspective view of the colpotomy ring;

FIG. 8b illustrates a close-up perspective view of the colpotomy ring;

FIG. 9a illustrates a perspective rear view of the colpotomy cup assembly;

FIG. 9b illustrates a perspective rear view of the colpotomy cup assembly, including a uterine sound;

FIG. 9c illustrates a perspective view of a locking mechanism for the uterine sound in the colpotomy cup assembly;

FIG. 9d illustrates a perspective view of the locking mechanism with the uterine sound in position for locking relative to the colpotomy cup assembly;

FIG. 10a illustrates a perspective view of electrocautery wire and tensioner cable attachment portions of the laparoscopic shaft assembly, including sliding handles;

FIG. 10b illustrates an exploded perspective view of the electrocautery wire and tensioner cable attachment portions;

FIG. 10c illustrates a perspective view of sliding attachments for the electrocautery wire and tensioner cable with the electrocautery wire and tensioner cable attached;

FIG. 11a is a perspective view of the laparoscopic shaft assembly;

FIG. 11b is a perspective view of the electrocautery wire and tensioner cable attachment portions, including a locking release button;

FIG. 12a is a perspective view of an electrocautery machine attached to the laparoscopic shaft assembly and FIG. 12b is a close-up view of the laparoscopic shaft assembly handle;

FIG. 13a is a perspective view of an electrocautery machine attached to an alternative embodiment laparoscopic shaft assembly;

FIG. 13b is a perspective view of a handle portion of the alternative embodiment laparoscopic shaft assembly; and

FIG. 14 is a perspective view of the uterine colpotomy system assembly and the laparoscopic shaft assembly being used to cut through a cervicovaginal junction to separate a cervix from a vagina.

### COMPONENT LISTING

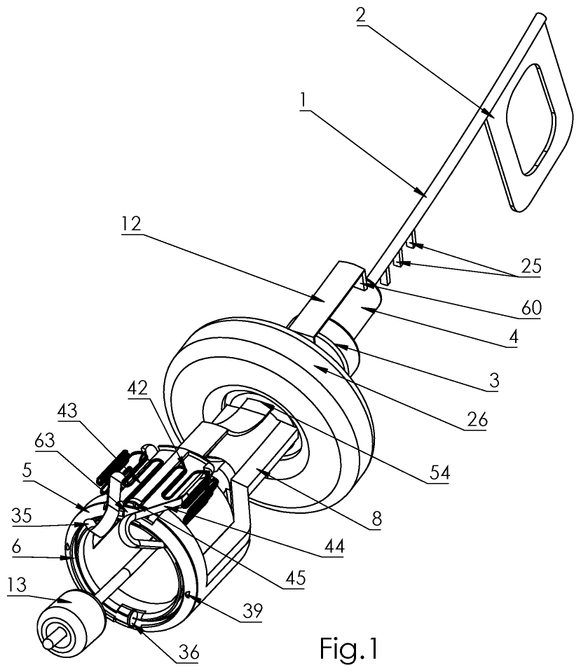

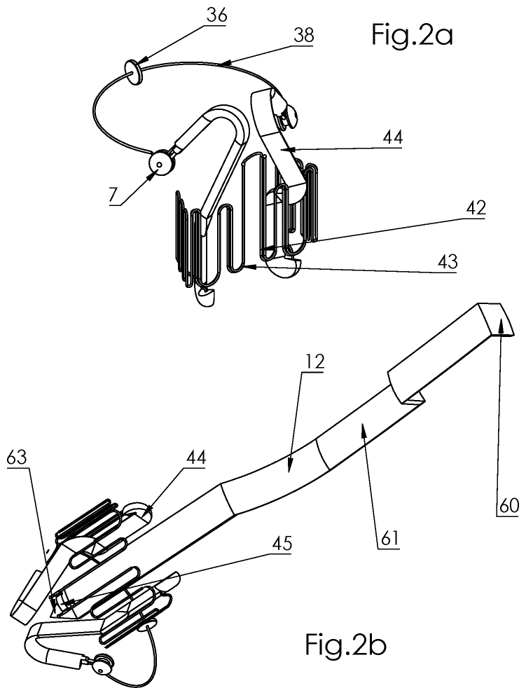

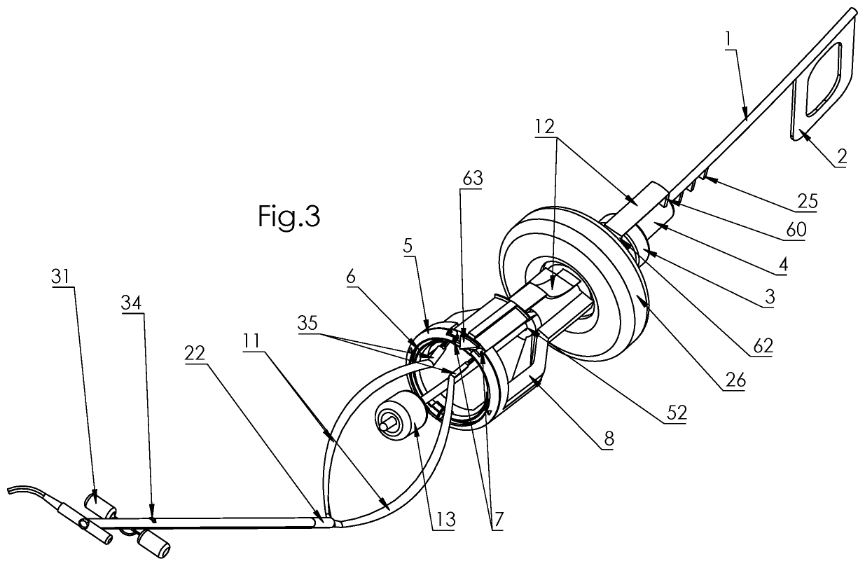

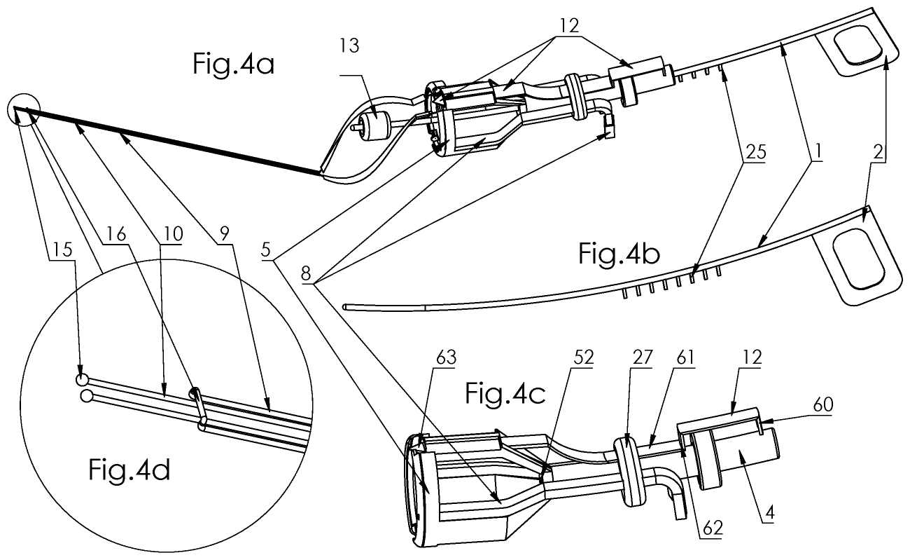

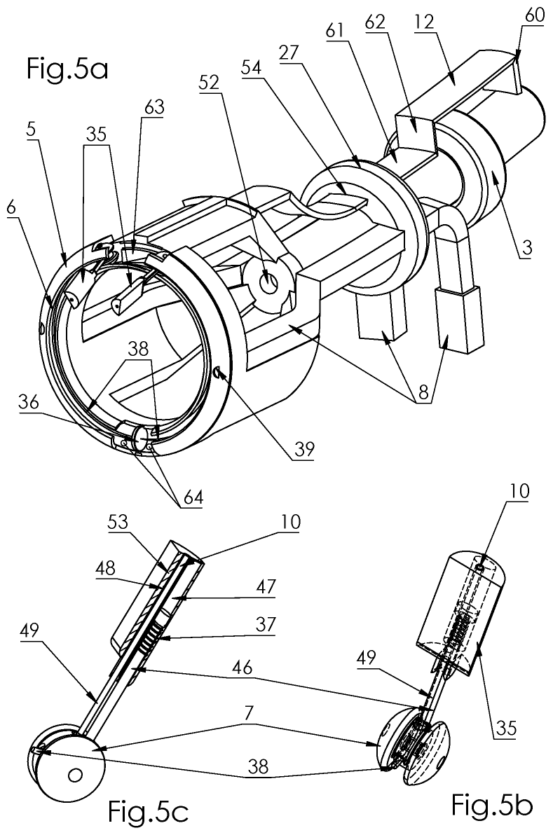

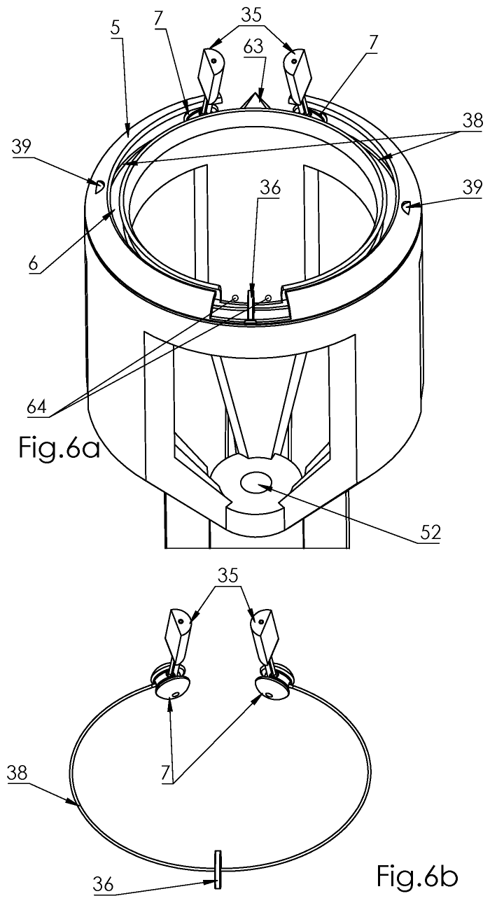

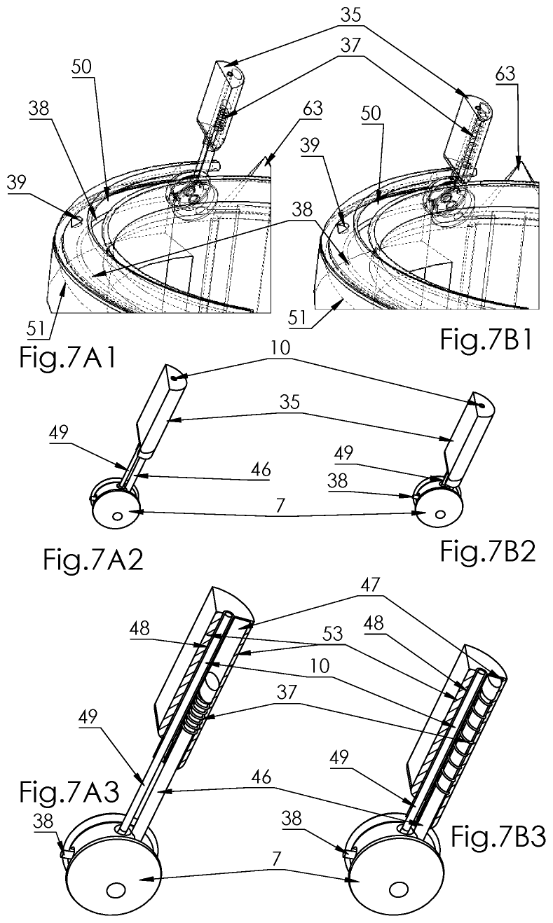

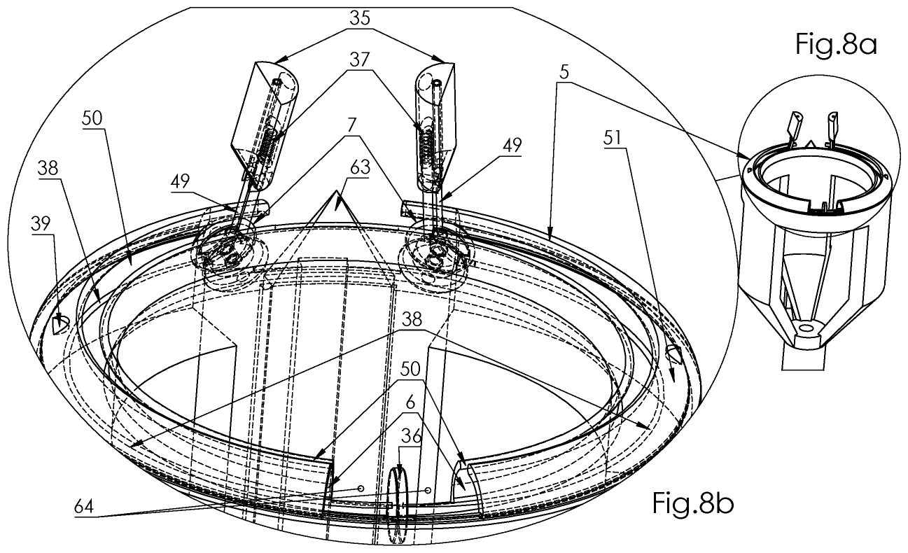

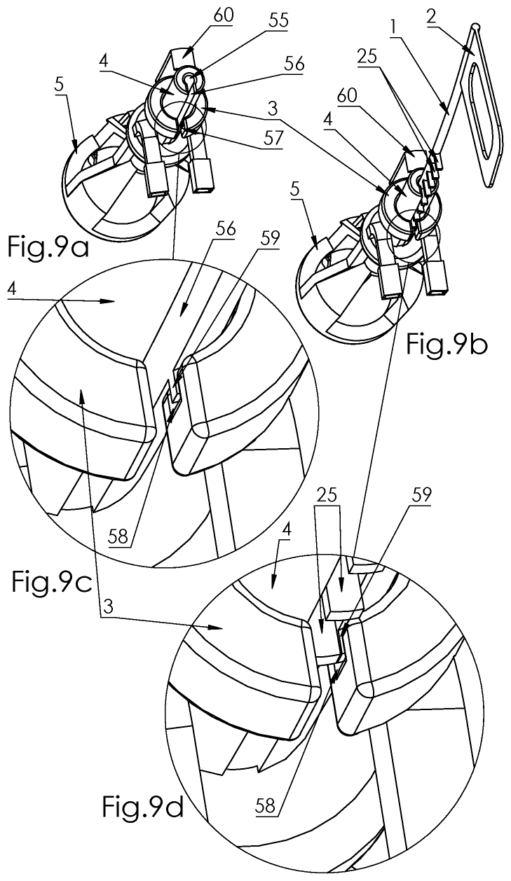

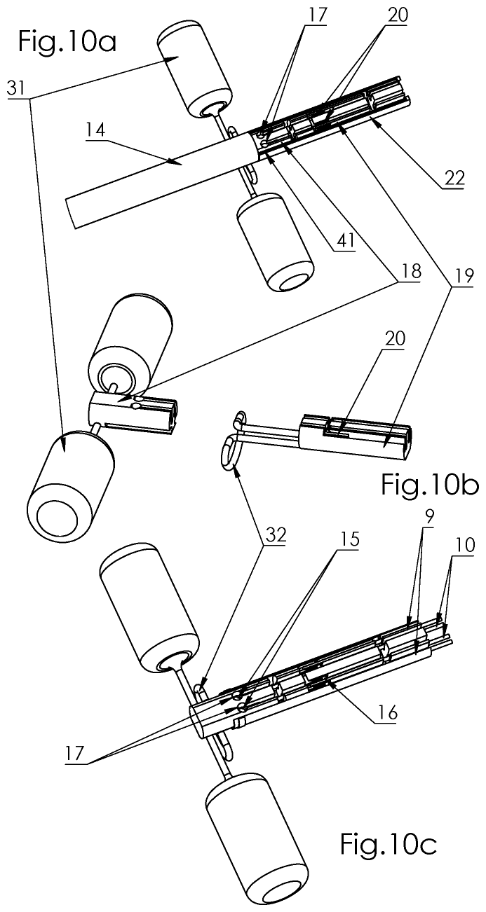

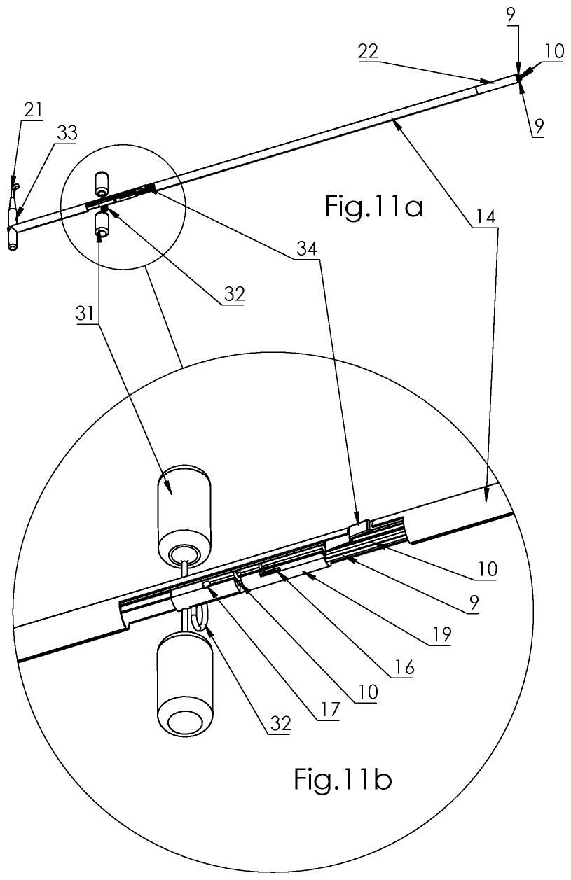

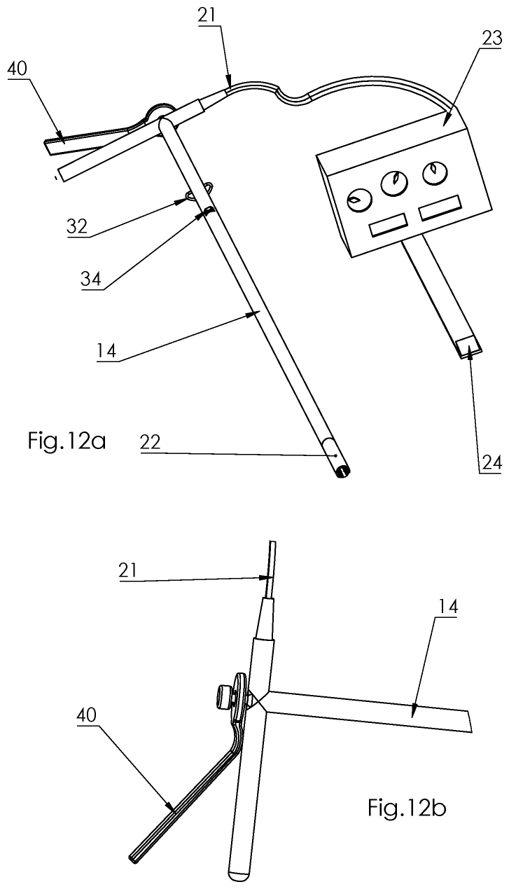

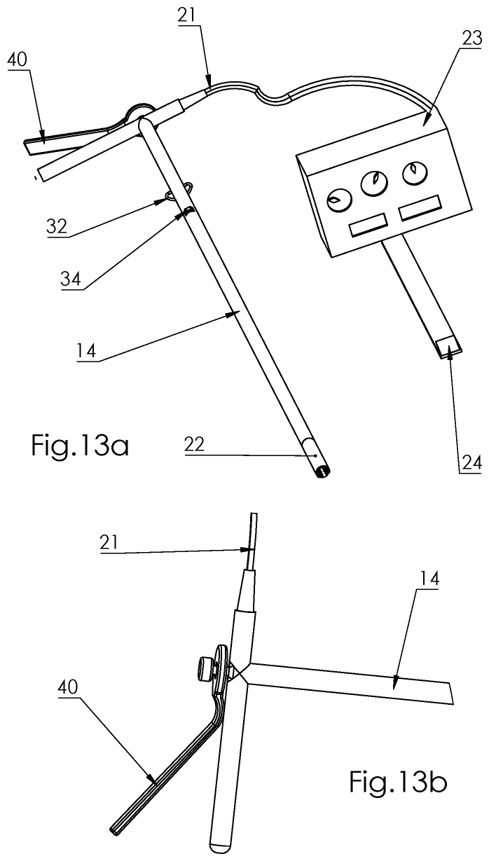

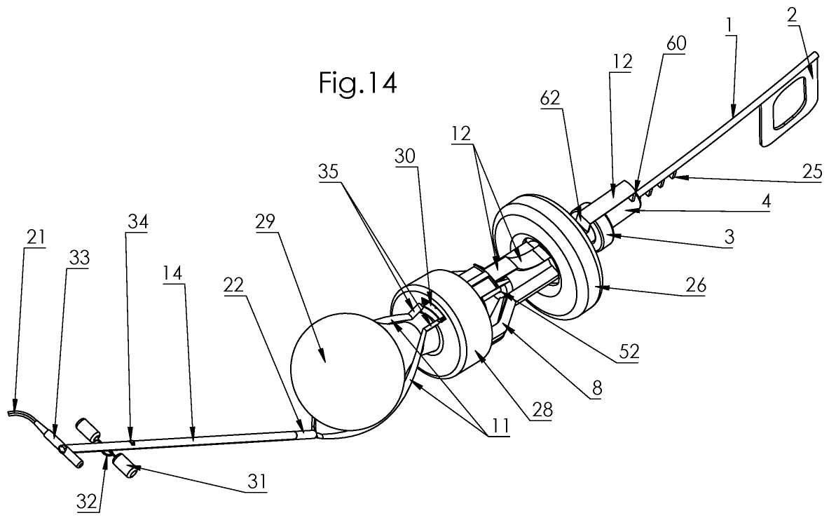

1. Uterine sound [see FIG. 1, Sheet 1/14; FIG. 3, Sheet 3/14; FIGS. 4a and 4b, Sheet 4/14; FIG. 9b, Sheet 9/14; and FIG. 14, Sheet 14/14].
2. Uterine sound handle [see FIG. 1, Sheet 1/14; FIG. 3, Sheet 3/14; FIGS. 4a and 4b, Sheet 4/14; FIG. 9b, Sheet 9/14; and FIG. 14, Sheet 14/14]
3. Locking ring to adjust uterine sound intrauterine length [see FIG. 1, Sheet 1/14; FIG. 3, Sheet 3/14; FIG. 5a, Sheet 5/14; FIGS. 9a, 9b, 9c and 9d, Sheet 9/14; and FIG. 14, Sheet 14/14].
4. Colpotomy cup cylinder [see FIG. 1, Sheet 1/14; FIG. 3, Sheet 3/14; FIG. 4c, Sheet 4/14; FIGS. 9a, 9b, 9c and 9d, Sheet 9/14; and FIG. 14, Sheet 14/14].
5. Colpotomy cup [see FIG. 1, Sheet 1/14; FIG. 3, Sheet 3/14; FIGS. 4a and 4c, Sheet 4/14; FIG. 5a, Sheet 5/14; FIG. 6a, Sheet 6/14; FIGS. 8a and 8b, Sheet 8/14; and FIGS. 9a and 9b, Sheet 9/14].
6. Colpotomy cup channel (tilted interior) [see FIG. 1, Sheet 1/14; FIG. 3, Sheet 3/14; FIG. 5a, Sheet 5/14; FIG. 6a, Sheet 6/14; and FIG. 8b, Sheet 8/14].
7. Colpotomy cup roller [see FIG. 2a, Sheet 2/14; FIG. 3, Sheet 3/14; FIGS. 5b and 5c, Sheet 5/14; FIGS. 6a and 6b, Sheet 6/14; FIGS. 7A2, 7B2, 7A3, 7B3, Sheet 7/14; and FIG. 8b, Sheet 8/14].
8. Light optical system [see FIG. 1, Sheet 1/14; FIG. 3, Sheet 3/14; FIGS. 4a and 4c, Sheet 4/14; FIG. 5a, Sheet 5/14; and FIG. 14, Sheet 14/14].
9. Tensioner cable handling insulated flexible sheath (Cable) which is attached permanently to one end of the flexible insulated sheath (11) [see FIGS. 4a and 4d, Sheet 4/14; FIG. 10c, Sheet 10/14; and FIGS. 11a and 11b, Sheet 11/14].
10. Electrocautery wire [see FIGS. 4a and 4d, Sheet 4/14; FIGS. 5c and 5b, Sheet 5/14; FIGS. 7A2, 7B2, 7A3, 7B3, Sheet 7/14; FIG. 10c, Sheet 10/14; and FIGS. 11a and 11b, Sheet 11/14].
11. Flexible insulated sheath (flexible sheath, insulated flexible sheath, insulated sheath, sheath) [see FIG. 3, Sheet 3/14; and FIG. 14, Sheet 14/14].
12. Introducer trocar that carries the electrocautery wire (10) and the tensioner cable (9) through the cervicovaginal junction [see FIG. 1, Sheet 1/14; FIG. 2b, Sheet 2/14; FIG. 3, Sheet 3/14; FIGS. 4a and 4c, Sheet 4/14; FIG. 5a, Sheet 5/14; and FIG. 14, Sheet 14/14].
13. Uterine sound intrauterine balloon or other uterine sound anchoring element to the uterus or intrauterine cavity [see FIG. 1, Sheet 1/14; FIG. 3, Sheet 3/14; and FIG. 4a, Sheet 4/14].
14. Laparoscopy shaft (Shaft) [see FIG. 10a, Sheet 10/14; FIGS. 11a and 11b, Sheet 11/14; FIGS. 12a and 12b, Sheet 12/14; FIGS. 13a and 13b, Sheet 13/14; and FIG. 14, Sheet 14/14].
15. Ball tips of the electrocautery wire (10) (Ball tips) [see FIGS. 4a and 4d, Sheet 4/14; and FIG. 10c, Sheet 10/14].
16. Horse shoe tip of the tensioner cable (9) handling the insulated Flexible sheath (11) [see FIGS. 4a and 4d, Sheet 4/14; FIG. 10c, Sheet 10/14; and FIG. 11b, Sheet 11/14].
17. Electrocautery ball socket to receive the Ball tips (15) of the electrocautery wire (10) [see FIGS. 10a and 10c, Sheet 10/14; and FIG. 11b, Sheet 11/14].
18. Sliding bar for electrocautery wire (10) hook up and constriction [see FIG. 10a, Sheet 10/14].
19. Sliding bar for tensioner cable (9) handling insulated Flexible sheath hook up and constriction (11) [see FIGS. 10a and 10b, Sheet 10/14; and FIG. 11b, Sheet 11/14].
20. Groove in the Sliding bar to hook up the Horse shoe tip of the tensioner cable (9) handling the insulated Flexible sheath (11) [see FIGS. 10a and 10b, Sheet 10/14; and FIG. 11b, Sheet 11/14].
21. Cable attachment to electrocautery Machine [see FIG. 11a, Sheet 11/14; FIGS. 12a and 12b, Sheet 12/14; FIGS. 13a and 13b, Sheet 13/14; and FIG. 14, Sheet 14/14]
22. Cover of the distal end of the laparoscopic shaft [see FIG. 3, Sheet 3/14; FIG. 10a, Sheet 10/14; FIG. 11a, Sheet 11/14; FIG. 12a, Sheet 12/14; FIG. 13a, Sheet 13; and FIG. 14, Sheet 14/14].
23. Electrocautery Machine [see FIG. 12a, Sheet 12/14; and FIG. 13a, Sheet 13].
24. Electrocautery Machine pedal to activate electrocautery machine with alternative activation button on the laparoscopy shaft handle (33) [see FIG. 12a, Sheet 12/14; and FIG. 13a, Sheet 13].
25. Uterine sound projections [see FIG. 1, Sheet 1/14; FIG. 3, Sheet 3/14; FIGS. 4a and 4b, Sheet 4/14; FIGS. 9b and 9d, Sheet 9/14; and FIG. 14, Sheet 14/14].
26. Vaginal occluder balloon with alternative other gas seal vaginal elements [see FIG. 1, Sheet 1/14; FIG. 3, Sheet 3/14; and FIG. 14, Sheet 14/14].
27. Vaginal occluder balloon base [see FIG. 4b, Sheet 4/14; and FIG. 5a, Sheet 5/14].
28. Vaginal vault [see FIG. 14, Sheet 14/14].
29. Uterus [see FIG. 14, Sheet 14/14].
30. A cut in the vaginal vault made by the introducer (12) to sever vagina from cervix [see FIG. 14, Sheet 14/14].
31. Handle for Sliding bar (18) for electrocautery wire hook up and constriction [see FIG. 3, Sheet 3/14; FIGS. 10a and 10b, Sheet 10/14; FIGS. 11a and 11b, Sheet 11/14; and FIG. 14, Sheet 14/14].
32. Handle for Sliding bar (19) for tensioner cable (9) handling insulated Flexible sheath (11) hook up and constriction [see FIGS. 10b and 10c, Sheet 10/14; FIGS. 11a and 11b, Sheet 11/14; FIG. 12a, Sheet 12/14; and FIG. 13a, Sheet 13; and FIG. 14, Sheet 14/14].
33. Handle for laparoscopy shaft [see FIG. 11a, Sheet 11/14; and FIG. 14, Sheet 14/14].
34. Release button for releasing locking system for Sliding bar (19) for tensioner cable (9) handling insulated Flexible sheath (11) [see FIG. 3, Sheet 3/14; FIGS. 11a and 11b, Sheet 11/14; FIG. 12a, Sheet 12/14; and FIG. 13a, Sheet 13; and FIG. 14, Sheet 14/14].
35. Release system of electrocautery wire at distal end of insulated Flexible sheath (11) [see FIG. 1, Sheet 1/14; FIG. 3, Sheet 3/14; FIGS. 5a and 5b, Sheet 5/14; FIGS. 6a and 6b, Sheet 6/14; FIGS. 7A1, 7B1, 7A2, 7B2, Sheet 7/14; FIG. 8b, Sheet 8/14; and FIG. 14, Sheet 14/14].
36. Electrocautery disc for supporting the electrocautery wire (10) with its folded configuration (43) and allows for both bipolar and monopolar electrocautery [see FIG. 1, Sheet 1/14; FIG. 2a, Sheet 2/14; FIG. 5a, Sheet 5/14; FIGS. 6a and 6b, Sheet 6/14; and FIG. 8b, Sheet 8/14].
37. Spring [see FIG. 5c, Sheet 5/14; FIGS. 6a and 6b, Sheet 6/14; FIGS. 7A1, 7B1, 7A3, 7B3, Sheet 7/14; and FIG. 8b, Sheet 8/14].
38. The uninsulated portion of Electrocautery wire (10) in colpotomy cup channel (6) [see FIG. 2a, Sheet 2/14; FIGS. 5a, 5b and 5c, Sheet 5/14; FIGS. 6a and 6b, Sheet 6/14; FIGS. 7A1, 7B1, 7B2, 7A3, 7B3, Sheet 7/14; and FIG. 8b, Sheet 8/14].
39. LED [see FIG. 1, Sheet 1/14; FIG. 5a, Sheet 5/14; FIG. 6a, Sheet 6/14; FIGS. 7A1 and 7B1, Sheet 7/14; and FIG. 8b, Sheet 8/14].
40. Alternative handle for manipulating Sliding bar (18) for electrocautery wire (10) hook up and constriction [see FIGS. 12a and 12b, Sheet 12/14; and FIGS. 13a and 13b, Sheet 13/14].
41. Ridge on laparoscopy shaft to guide Cover (22) at the distal end of the laparoscopic shaft (14) [see FIG. 10a, Sheet 10/14].
42. Folded Tensioner cable handling insulated flexible sheath (Tensioner cable (9) in accordion-folded configuration) [see FIG. 1, Sheet 1/14; FIG. 2a, Sheet 2/14].
43. Folded electrocautery wire (electrocautery wire (10) in accordion-folded configuration) [see FIG. 1, Sheet 1/14; FIG. 2a, Sheet 2/14].
44. Folded insulated Flexible sheath (insulated flexible sheath (11) in accordion-folded configuration) [see FIG. 1, Sheet 1/14; FIGS. 2a and 2b, Sheet 2/14].
45. A temporary attachment between the electrocautery wire 10,43, the cable 9, 42 and the introducer trocar 12 [see FIG. 1, Sheet 1/14; FIG. 2b, Sheet 2/14].
46. Roller rod [see FIGS. 5b and 5c, Sheet 5/14; and FIGS. 7A2, 7A3 and 7B3, Sheet 7/14].
47. Roller rod channel in release system (35) of electrocautery wire (10) at distal end of insulated Flexible sheath (11) [see FIG. 5c, Sheet 5/14; and FIGS. 7A3 and 7B3, Sheet 7/14].
48. Electrocautery duct in insulated Flexible sheath (11) [see FIG. 5c, Sheet 5/14; and FIGS. 7A3 and 7B3, Sheet 7/14].
49. Exposed portion of electrocautery wire (10) with its continuity (38) in colpotomy cup channel (6) [see FIGS. 5b and 5c, Sheet 5/14; and FIGS. 7A2, 7B2, 7A3 and 7B3, Sheet 7/14; and FIG. 8b, Sheet 8/14].
50. Top opening of the Colpotomy cup channel [see FIGS. 7A1 and 7B1, Sheet 7/14; and FIG. 8b, Sheet 8/14].
51. Floor of the Colpotomy cup channel (tilted interior) [see FIGS. 7A1 and 7B1, Sheet 7/14; and FIG. 8b, Sheet 8/14].
52. Distal opening of the Colpotomy cup cylinder (4) duct through which the uterine sound is housed when the device is in use.
53. Cut face of distal end of insulated Flexible sheath [see FIG. 5c, Sheet 5/14; and FIGS. 7A3 and 7B3, Sheet 7/14].
54. Groove for the Introducer trocar (12) for electrocautery wire (12) within the vaginal occluder balloon base (27) [see FIG. 1, Sheet 1/14; and FIG. 5a, Sheet 5/14].
55. Proximal opening of the Colpotomy cup cylinder (4) duct through which the uterine sound is housed when the device is in use [see FIG. 9a, Sheet 9/14].
56. Groove in the colpotomy cup cylinder through which uterine sound projections (25) can pass [see FIGS. 9a and 9c, Sheet 9/14].
57. Slit opening in the locking ring (3) [see FIG. 9a, Sheet 9/14].
58. Groove in the locking ring (3) that encase the locked uterine sound projection when the locking ring (3) is turned [see FIGS. 9c and 9d, Sheet 9/14].
59. Ridge on the colpotomy cup cylinder to guide the locking ring [see FIGS. 9c and 9d, Sheet 9/14].
60. The introducer trocar (12) handle tab [see FIG. 1, Sheet 1/14; FIG. 2b, Sheet 2/14; FIG. 3, Sheet 3/14; FIG. 4c, Sheet 4/14; FIG. 5a, Sheet 5/14; FIGS. 9a and 9b, Sheet 9/14; and FIG. 14, Sheet 14/14].
61. Introducer trocar (12) off-set portion [see FIG. 2b, Sheet 2/14; FIG. 4c, Sheet 4/14; and FIG. 5a, Sheet 5/14].
62. Introducer trocar 12 off-set face [see FIG. 3, Sheet 3/14; FIG. 4c, Sheet 4/14; FIG. 5a, Sheet 5/14; and FIG. 14, Sheet 14/14].
63. Introducer trocar 12 pointing tip or a blade [see FIG. 1, Sheet 1/14; FIG. 2b, Sheet 2/14; FIG. 3, Sheet 3/14; FIG. 4c, Sheet 4/14; FIG. 5a, Sheet 5/14; FIG. 6a, Sheet 6/14; FIGS. 7A1and 7B1, Sheet 7/14; and FIG. 8b, Sheet 8/14].
64. Colpotomy cup (5) opening for releasing colpotomy cup rollers (7) [see FIG. 5a, Sheet 5/14; FIG. 6a, Sheet 6/14; and FIG. 8b, Sheet 8/14].
65. Button activating electrocautery [see FIG. 14, Sheet 14/14].

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

Referring to FIG. 1, a colpotomy system assembly is shown. The colpotomy system assembly includes a uterine sound 1 and a handle 2. A locking ring 3 is provided on the uterine sound 1, the locking ring 3 engaging one of a series of uterine sound projections 25 for adjusting and locking the uterine sound 1 in position relative to the a colpotomy cup 5 at the proper intrauterine length.

A colpotomy cup cylinder 4 surrounds the uterine sound 1 around a portion of its length, and supports the colpotomy cup 5. The colpotomy cup 5 has a forward facing toroidal profile and includes a colpotomy cup channel 6. The colpotomy cup channel 6 is disposed at a slightly inward angle along the toroidal surface. A light optical system 8 extends along the colpotomy cup cylinder 4, and extends to the colpotomy cup 5 to illuminate the area around the colpotomy cup 5, and may include one or more LEDs 39 to provide effective illumination of the vagina and vaginal fornices during the introduction of the colpotomy cup around the cervix and to transilluminate through the cervicovaginal junction and the surrounding tissue to help during the laparoscopic tissues dissection and avoid injury to blood vessels and surrounding organs.

An introducer trocar 12 is provided for introducing an electrocautery wire 10, shown here in an accordion-folded configuration 43, as well as a cable 9, also shown here in an accordion-folded configuration 42 from the vagina into the intrauterine cavity through the cervicovaginal junction, and allowing the electrocautery wire 43 and cable 42 to be grasped by a regular laparoscopic grasper (not shown) and coupled to a laparoscopy shaft assembly (See, FIGS. 3, 4a, 11a, 12 and 14). The introducer trocar 12 extends through an introducer trocar groove 54 located in a vaginal occluder balloon base 27 which supports a vaginal occluder balloon 26. A uterine sound intrauterine balloon 13 is provided near a terminal end of the uterine sound 1 opposite the uterine sound handle 2 which help to stabilize and anchor the distal end of the uterine sound to the uterus and the uterine cavity.

Additional components of the colpotomy system assembly discussed in more detail below include release systems 35 for the electrocautery wire 10, 43 located at a distal end of insulated flexible sheaths 11, 44. An electrocautery disc 36 is provided for supporting the electrocautery wire 10, 43 and allows for both bipolar and monopolar electrocautery. A temporary attachment 45 is provided between the electrocautery wire 43, the cable 42 and the introducer trocar 12.

Referring to FIGS. 2a and 2b, the electrocautery portions of the colpotomy system assembly are shown in more detail. Referring to FIG. 2a, the cable 42 and electrocautery wire 43 are preferably accordion folded together. While the cable 9, 42 is attached to the proximal end of the insulated sheaths 44, the electrocautery wire 10, 43 extends through the insulated sheaths 44, continuing along roller rods 46 and through colpotomy cup rollers 7 before traveling around the colpotomy cup 5 in the colpotomy cup channel 6 (not shown). The electrocautery wire 10, 43 includes an uninsulated portion 38 disposed in the colpotomy cup channel 6, which terminates at the electrocautery disc 36.

In an alternative embodiment, the folded electrocautery wire 43, the folded insulated flexible sheaths 44 and the folded cable 42 are enclosed in a sterile encasement (not shown) to protect them from being contaminated from the different microbes that are present in the vagina prior to their introduction into the intra-abdominal cavity.

Referring to FIG. 2b, The introducer trocar 12 is shown interfacing with the electrocautery wire 43 and cable 42. The introducer trocar 12 includes a handle portion 60 for engagement by the surgeon, to push the introducer trocar 12 forward relative to the colpotomy cup 5 (not shown). Continued forward movement of the introducer trocar 12 may be arrested by providing a handle tab 60 configured to catch on the locking ring 3. The introducer trocar 12 also includes an off-set portion 61 allowing it to travel through the vaginal occluder balloon base 27 and not interfere with the vaginal occluder balloon 26 once inflated. The off-set portion 61 may include an off-set face 62 configured to abut the vaginal occluder balloon base 27 when the handle tab 60 engages the locking ring 3. The introducer trocar 12 also preferably includes a pointing tip or a blade 63 for piercing the vaginal fornix and the cervicovaginal junction tissue. The electrocautery wire 43 and cable 42 are preferably semi-permanently attached to the introducer trocar 12 such that they are carried from the vagina through the vaginal fornix and the cervicovaginal junction along with its tip 63 in order to be grasped by a regular laparoscopic grasper in the intra-abdominal side and manually or in a different embodiment automatically coupled with the laparoscopy shaft assembly.

Referring to FIG. 3, the colpotomy system assembly is shown with cable 9, the electrocautery wire 10, and the flexible sheaths 11 extended into the laparoscopy shaft assembly. The laparoscopy shaft assembly includes a cover 22 distal from the laparoscopy shaft handle 33. The laparoscopy shaft assembly also includes an electrocautery wire sliding bar handle 31 pulling the electrocautery wire 10 through the laparoscopy shaft assembly and a cable sliding bar handle 32 for pulling the cable/tensioner cable 9 through the laparoscopy shaft assembly. A release button 34 is provided for releasing the cable sliding bar handle 32. Also shown in this view, opposite the cover 22, the flexible sheaths 11 each comprise a release system 35 for controlling the exposure of the uninsulated portion of the electrocautery wire to the cervicovaginal junction tissue.

Referring to FIGS. 4a-4d, details of the colpotomy system assembly are shown, including the uterine sound 1 and uterine sound handle 2 disposed opposite the uterine sound intrauterine balloon 13. The colpotomy cup cylinder 4 is also shown, including the introducer trocar 12, vaginal occluder balloon base 27 and the colpotomy cup cylinder distal opening 52 through which the uterine sound 1 passes. In this view the cable/tensioner cable handling insulated flexible sheath 9 is shown along with the insulated electrocautery wire 10. The insulated electrocautery wire 10 includes ball tips 15 for engaging the laparoscopy shaft assembly, while the cable/tensioner cable handling insulated flexible sheath 9 ends in a horseshoe tip for the same purpose.

Referring to FIGS. 5a-5c, the release systems 35 for the electrocautery wire 10 with its portion 38 located in colpotomy cup channel 6 are shown in more detail. As shown in FIG. 5b and in the partial cross section in FIG. 5c, each release mechanism 35 includes a colpotomy cup roller 7 that travels around the colpotomy cup channel 6. Specifically, FIG. 5c shows a cut through the distal end of the insulated flexible sheath 53 that shows the cut face of the distal end of the insulated flexible sheath 53. A roller rod 46 extends from each colpotomy cup roller 7, extending into a roller rod channel 47. A spring 37 is housed in each roller rod channel 47, and an electrocautery duct 48 is provided in each insulated flexible sheath 11 such that the exposed portion electrocautery wire 49 can travel through the insulated flexible sheaths 11 and into the laparoscopy shaft assembly. The exposed portion electrocautery wire 49 also extends through the colpotomy cup rollers 7, which guide its movement around the colpotomy cup 5.

Referring to FIGS. 6a and 6b, the placement of the electrocautery wire 38 in the colpotomy cup channel 6 is shown. The electrocautery wire 38 terminates in an electrocautery disc 36, thus allowing for bipolar electrocautery if the disc 36 prevents the transmission of electricity between the electrocautery wire on both sides of the disc 36, and alternatively, for monopolar electrocautery if it allows the transmission of electricity between the electrocautery wire on both sides of the disc 36. As shown in this view the introducer trocar 12 is located adjacent the release systems 35 in their starting positions. Once the colpotomy cup rollers 7 have travelled the circumference of the colpotomy cup channel 6 and reached the electrocautery disc 36, the electrocautery wire 10 and the cable 9 are uncoupled from the laparoscopy shaft assembly and the colpotomy system assembly will be removed from the vagina along with the severed cervix and the uterus, or in a different embodiment the colpotomy cup rollers 7 are released from the colpotomy cup 5 by an opening in the colpotomy cup channel 6 adjacent to the electrocautery disc 36 and they are removed from the body through the laparoscopic port along with the electrocautery wire 10, the cable 9 and the laparoscopy shaft assembly.

Referring to FIGS. 7a1 through 7b3, the release systems 35 are shown in their extended (7a1, 7a2 and 7a3) and contracted (7b1, 7b2 and 7b3) states. Referring to FIGS. 7a1 and 7b1, the uninsulated portion fo the electrocautery wire 38 travels along a floor 51 of the colpotomy cup channel 50. Referring to FIGS. 7b1, 7b2 and 7b3, when the cable handling insulated flexible sheath 9 is not under tension, the spring 37 causes the insulated sheath 11 to extend downward along the roller rod 46, substantially covering the exposed portion electrocautery wire 49 in the process. When a user pulls the cable 9, the insulated sheaths 11 are pulled upward along the roller rods, compressing the spring 37 in each roller rod channel 47, and exposing the exposed portion electrocautery wire 49 between the colpotomy cup rollers 7 in the colpotomy cup channel 6. The exposed portion electrocautery wire 49 is located in front of the roller rod 46 in the direction of travel of each colpotomy cup roller to facilitate cutting.

Referring to FIGS. 8a and 8b, a detail of the colpotomy cup 5 is shown. In this view the release systems 35 are in their retracted state, exposing the electrocautery wire 38 between the insulated sheaths 11 and the colpotomy cup rollers. Also, as shown in this view, the colpotomy cup channel 6 comprises a semicircular channel having an opening 50 through which the electrocautery wire 38, but not the colpotomy cup rollers 7 can pass. The opening is oriented slightly inward, angled more toward the cervix (not shown) and away from the surrounding organs (not shown) to decrease the risk of injury of the surrounding organs by the exposed electrocautery wire 49. Also shown in this view is the electrocautery disc 36, and adjacent spaces through which the colpotomy cup rollers 7 pass when disengaging from the colpotomy cup 5. Preferably, the channel 6 may comprise a translucent material such that the LEDs 39, or similar lighting system illuminate area adjacent the colpotomy cup 5.

Referring to FIGS. 9a-9d, operation of the locking ring 3 to adjust the uterine sound intrauterine length is shown in detail. FIG. 9a shows the colpotomy cup cylinder 4 and colpotomy cup 5, including the proximal opening of the colpotomy cup cylinder duct 55 through which the uterine sound 1 extends when in use. The colpotomy cup cylinder 4 includes a groove 56 through which the uterine sound projections 25 can pass as the uterine sound 1 travels through the colpotomy cup cylinder 4. Additionally, the locking ring 3 includes a slit opening 57 coinciding with the groove 56. A ridge 59 on the colpotomy cup cylinder 4 guides the locking ring 3 with a groove 58 in the locking ring 3, enabling the uterine sound 1 to be locked in position using the uterine sound projections 25 as shown in FIG. 9d.

Referring to FIGS. 10a-10c, the proximal portion of the laparoscopy shaft assembly is shown. The laparoscopy shaft 14 includes a ridge 41 for guiding the cover 22 at the end of the laparoscopy shaft 14. A pair of electrocautery ball sockets 17 are provided for receiving the ball tips 15 of the electrocautery wire 10. A first sliding bar 18 is provided to connect and constrict the electrocautery wire 10. A second sliding bar 19 is provided for connecting the cable 9 which is connected to the insulated sheaths 11. A groove 20 is provided in the second sliding bar 19 for receiving the horse shoe tip 16 of the cable 9 handling the insulated flexible sheaths 11. As shown in FIG. 10c, the horseshoe tip 16 of the cable 9 is installed in the groove 20, which may be manipulated by operating the cable sliding bar handle 32, while the electrocautery wire sliding bar handles directly control the electrocautery wire 10 using the ball tips 15.

FIGS. 11a and 11b show an enlarged view of the laparoscopy shaft assembly controls. In particular, the release button 34 is shown enabling a user to release the cable sliding bar handle 32 after it has been pulled to take up all of the cable 9, such that the insulated flexible sheaths 11 are abutting the distal end of the laparoscopy shaft 14. The electrocautery ball sockets 17 and first sliding bar 18 are also shown, which are controlled by the electrocautery wire sliding bar handle 31. The laparoscopy shaft handle 33 is connected to a cable attachment to an electrocautery machine 23 (FIG. 12).

Referring to FIGS. 12a and 12b, the electrocautery machine 23 is shown, including an electrocautery machine pedal 24. The electrocautery machine 23 is connected to the cable attachment 21 and laparoscopy shaft handle 33. With the electrocautery machine 23 connected to the laparoscopy shaft assembly, and the cable sliding bar handle 32 retracted until the release button 34 catches it, the electrocautery wire sliding bar handle 31 may be retracted, or otherwise manipulated, to pull the electrocautery wire 10 with its uninsulated portion located in colpotomy cup channel 38 around the colpotomy cup 5.

Referring to FIGS. 13a and 13b, an alternative laparoscopy shaft assembly is shown featuring an alternative handle 40 for manipulating the first sliding bar 18 and manipulating the electrocautery wire. In this embodiment, compression of the alternative handle 40 against the laparoscopy shaft handle 33 causes tension in the first sliding bar 18 and ball tips 15 and thus pulls the electrocautery wire 38 from the colpotomy cup channel 6.

FIG. 14 shows the colpotomy system assembly and the laparoscopy shaft assembly in use separating a vagina at the vaginal vault, represented by cylinder 28, from a uterus, represented by sphere 29. In this view, the uterine sound assembly is inserted into the vagina such that the colpotomy ring 5 abuts the vaginal vault 28. This is done after sounding the uterus 29, and locking the uterine sound 1 in position using the uterine sound projections 25 and the locking ring 3. The vaginal occluder balloon 26 is inflated, although shown in a relatively smaller diameter in this view for illustration purposes.

Still referring to FIG. 14, the introducer trocar 12 has been pushed forward from the vagina through the vaginal vault 28 and the cervicovaginal junction, carrying with it, the ball tips 15 of the electrocautery wire 10 in its folded state 43, and the horseshoe tip 16 of the cable 9 in its folded state 42. In the process the folded electrocautery wire 43 and cable 42 have unfolded and travelled through the cut 30 in the vaginal fault 28 into the intra-abdominal side which are then grasped by a regular laparoscopic grasper (not shown) and manually coupled with the laparoscopy shaft assembly after bringing them out of the abdomen through the laparoscopy port (not shown) or in a different embodiment automatically coupled with the laparoscopy shaft assembly.

The user has moved the electrocautery bar handle 31 and the cable sliding bar handle 32 back until the release button 34 has caught, preventing the cable sliding bar handle 32 from moving in a reverse direction. At which time the insulated flexible sheaths 11 are abutting the distal end of the laparoscopy shaft 14 and they both move as one unit. Pulling the laparoscopy shaft 14 causes pulling on the insulated flexible sheath which in turn cause the release systems 35 to be activated, exposing the electrocautery wire 49. Once the electrocautery machine 23 is activated, the exposed electrocautery wire 49 will heat up and cut the vaginal vault 28 as the electrocautery wire sliding bar handle 31 is pulled, the electrocautery wire 10 is pulled causing the uninsulated portion of the electrocautery wire in the colpotomy channel 38 to shorten, causing the colpotomy cup rollers 7 to travel around the colpotomy cup channel 6 of the colpotomy cup 5, which is pressed against the vaginal vault 28, thereby severing it from the uterus 29.

Once the release systems 35 reach the electrocautery disc 36, the electrocautery wire 10 and the cable 9 are uncoupled from the laparoscopy shaft assembly and the colpotomy system assembly will be removed from the vagina along with the severed cervix and the uterus, or in a different embodiment, the colpotomy cup rollers 7 are released from the colpotomy cup 5 by an opening 64 in the colpotomy cup channel 6 adjacent the electrocautery disc 36 and they are removed from the body through the laparoscopic port along with the electrocautery wire 10, the cable 9 and the laparoscopy shaft assembly.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

1. A device to facilitate a total laparoscopic hysterectomy wherein a uterus and a portion of a cervix are inside an intra-abdominal cavity and a vagina circumferentially attached to the cervix creating a cervicovaginal junction, wherein the cervix has a cervical canal, the uterus has an intrauterine cavity, and the intra-abdominal cavity includes an access installed through the anterior abdominal wall, the device comprising:
a cervical cup with a cervical cup proximal end and a cervical cup distal end, the cervical cup distal end adapted to embrace the cervix with the cervical cup distal end abutting the cervicovaginal junction, and the cervical cup proximal end extending away from the cervix;
an electrocathery wire having an electrocautery wire proximal end and an electrocathery wire distal end, wherein the electrocautery wire distal end includes an uninsulated portion;
the cervical cup distal end having a channel housing the uninsulated portion, the channel configured for controlled release of the unillsulated portion of the electrocautery wire distal end;
an introducer having an introducer proximal end and an introducer distal end, the introducer proximal end releasably coupled to the electrocautery wire proximal end;
wherein the introducer is configured to penetrate the cervicovaginal junction upon activation of the introducer distal end, thereby introducing the electrocauter wire proximal end into the intra-abdominal cavity;
the device further comprising a shaft having a shaft proximal end, and a shaft distal end, wherein the shaft distal end is configured for introduction into the intra-abdominal cavity through the access installed in the anterior abdominal wall;
wherein the shaft is further configured to secure the electrocautery wire proximal end through the shaft distal end and move the electrocautery wire proximal end through the shaft toward the shaft proximal end, thereby pulling the electrocautery wire uninsulated portion from the channel and causing a controlled release of the uninsulated portion along a predetermined circumferential path around the cervix along the channel; and
wherein the vagina is severed from the cervix along the circumference of the cervicovaginal junction.

2. The device of claim 1 further comprising two sliders inside the channel, wherein each slider is configured to slide in opposing 180 degree semi-circular tracks until the two sliders meet; wherein each slider has a duct having an inlet and an outlet, and wherein the uninsulated portion of the electrocautery wire passes through the duct of each slider from the inlet to the outlet, emerging from each outlet toward an outside of the channel, thereby allowing the uninsulated portion to be released along the channel and the electrocautery wire to disengage the cervical cup through the outlet of each duct.

3. The device of claim 2 further comprising two flexible insulated sheaths, wherein each flexible insulated sheath has a sheath proximal end and a sheath distal end, wherein each sheath distal end is individually coupled to a slider such that each flexible insulated sheath can move a predetermined distance toward or away from each slider, wherein each flexible insulated sheath includes a duct having a duct proximal end and a duct distal end, wherein each duct distal end faces each slider and each duct proximal end is releasably coupled to the shaft distal end, wherein each of the ducts house a portion of the electrocautery wire, with the electrocautery wire extended from the duct distal end to the duct proximal end, and wherein the flexible insulated sheaths are configured such that pulling on the proximal end of the shaft with the shaft distal end coupled to each flexible insulated sheath causes each of the flexible insulated sheaths to move away from each slider for a predetermined distance exposing portion of the uninsulated electrocautery wire; and wherein the electrocautery wire is configured such that moving the proximal end through the long shaft causes the electrocautery wire to move through the duct in a manner that the uninsulated portion extends only between a slider and a flexible insulated sheath, thereby causing the uninsulated portion to slide through a predetermined circular path around the cervix.

4. The device of claim 1 further comprising an elongated curved stem having a stem proximal end, a stem distal end and a first curved axis, the stem distal end adapted to be introduced through the cervical canal into the intrauterine cavity thereby providing a means for manipulating the uterus, the proximal end having a handle; the device further comprising an elongated curved cylinder having a proximal end, a distal end, a duct along its length and a second curved axis matching the first curved axis, the elongated curved cylinder being shorter in length than the elongated curved stem, and wherein the distal end of the elongated cylinder is adapted to be coupled with the proximal end of the cervical cup thereby allowing them to move as one unit, wherein the elongated curved stem is configured for sliding through the duct of the elongated curved cylinder and adapted to be locked in place at a predetermined distance set by the operator.

5. The device of claim 1 further comprising a cable having a cable proximal end and a cable distal end, the cable distal end attached to the flexible insulated sheath proximal end and the cable proximal end configured for releasably coupling to the shaft distal end, thereby facilitating temporary coupling of the shaft with the insulated flexible sheath, and allowing them to move as a single unit; wherein the cable proximal end is configured to be releasably coupled to the introducer proximal end alongside the electrocautery wire proximal end, and wherein actuating the introducer distal end pushes the introducer forward, penetrating a small portion of the cervicovaginal junction, thereby introducing both the cable proximal end and the electrocautery wire proximal end to the inside of the intra-abdominal cavity.

6. The device of claim 2 wherein the semicircular tracks of the channel at the cervical cup distal end are tilted interiorly, thereby causing the sliders to be tilted interiorly along the track, and causing the angle of the exposed portion of the electrocautery wire extending between each slider and each flexible insulated sheath to be tilted interiorly closer to the cervix, thereby causing the cutting area severed by the uninsulated portion in the cervicovaginal junction to be closer to the cervix and away from other body organs.

7. The device of claim 1 wherein a portion of the shaft is flexible.

8. The device of claim 1 wherein a portion of the shaft is bifurcated.

9. The device of claim 1 wherein the shaft is insulated.

10. The device of claim 1 further comprising an optical/light system terminating at the cervical cup.

11. The device of claim 1 wherein the shaft includes sliding bars configured to move the electrocautery wire through the shaft.

12. The device of claim 1 wherein the uninsulated portion of the electrocautery wire distal end is divided into two arms, wherein each arm is coupled to an opposing side of a non-conductive element, thereby allowing the electrocautery wire to function using a bipolar electrocautery technique.

13. The device of claim 1 wherein the non-conductive element is anchored in place in the channel at the cervical cup distal end.

14. The device of claim 1 further comprising a component selected from the group consisting of a monopolar wired electrocautery energy generator, a bipolar wired electrocautery energy generator, a monopolar batteries operated electrocautery energy generator, a bipolar batteries operated electrocautery energy generator, a pedal activating electrocautery, a manual activating electrocautery, and a button activating electrocautery, a gas seal vaginal element, a gas seal vaginal balloon occluder, and at least one apparatus chosen from the list of handle, uterine sound's intrauterine balloon, and uterine sound's anchoring element to the uterus.

15. A device to facilitate a total laparoscopic hysterectomy wherein a uterus and a portion of a cervix are inside an intra-abdominal cavity and a vagina circumferentially attached to the cervix creating a cervicovaginal junction, wherein the cervix has a cervical canal, the uterus has an intrauterine cavity, and the intra-abdominal cavity includes an access installed through the anterior abdominal wall, the device comprising:
a cervical cup having a cervical cup distal end adapted to abut the cervicovaginal junction, and a cervical cup proximal end extending away from the cervix;
electrocautery wire having an insulated portion, and an uninsulated portion disposed in the cervical cup distal end;
the cervical cup distal end having a channel housing the uninsulated portion, the channel comprising a pair of sliders configured to move through the channel and release of the uninsulated portion of the electrocautery wire;
an introducer having an introducer proximal end and an introducer distal end, the introducer proximal end releasably coupled to the electrocautery wire insulated portion, the introducer slidable relative to the cervical cup such that the proximal end is configured to penetrate the cervicovaginal junction upon activation of the introducer distal end, thereby introducing the electrocautery wire insulated portion into the intra-abdominal cavity;
the device further comprising a shaft having a shaft proximal end, and a shaft distal end, wherein the shaft distal end is configured for introduction into the intra-abdominal cavity through the access installed in the anterior abdominal wall;
wherein the shaft is further configured to secure the electrocautery wire insulated portion through the shaft distal end and urge the electrocautery wire through the shaft toward the shaft proximal end, thereby pulling the electrocautery wire uninsulated portion through the sliders and around the channel, thereby causing a controlled release of the uninsulated portion along a predetermined circumferential path around the cervix.

16. A method of separating a patient's cervix from the patient's vagina at the cervicovaginal junction, the method comprising the steps of:
providing a colpotomy cup assembly having a colpotomy ring
providing an electrocautery wire on the colpotomy cup assembly;
passing the electrocautery wire through an insulated flexible sheath;
providing a tensioner cable on the insulated flexible sheath;
providing an introducer trocar on the colpotomy cup assembly in a sliding relationship to the colpotomy ring;
anchoring the electrocautery wire and the tensioner cable to the introducer trocar;
placing the colpotomy ring against the cervicovaginal junction;
urging the introducer trocar through the cervicovaginal junction, thereby carrying the electrocautery wire and tensioner cable through the cervicovaginal junction;
capturing the electrocautery wire and the tensioner cable with a laparoscopic shaft assembly;
placing the electrocautery wire and the tensioner cable under tension; and
pulling the electrocautery wire through the insulated flexible sheath while causing the electrocautery wire to travel around the colpotomy ring.

17. The method of claim 16 further comprising the step of providing a two sliders coupled to the two flexible insulated sheaths, the two sliders are configured to travel along with the flexible insulated sheaths around two semi-circular portions of the colpotomy ring and control the release of the electrocautery wire from the colpotomy ring.

18. The method of claim 16 further comprising the step of providing a first handle for moving the tensioner cable into the laparoscopic shaft assembly, such that the flexible insulated sheath abuts the laparoscopic shaft assembly.

19. The method of claim 16 further comprising the step of providing a second handle on the laparoscopic shaft assembly for drawing the electrocautery wire through the flexible insulated sheath.

20. The method of claim 16 further comprising the step of providing a release system comprising two colpotomy cup rollers, each colpotomy cup roller affixed to a roller rod, and urging the electrocautery wire through the colpotomy cup rollers, such that the colpotomy cup rollers travel around the colpotomy ring in opposite directions, providing a channel in the colpotomy ring, the channel configured to allow the colpotomy cup rollers to pass therethrough, and providing a roller rod channel and a spring in each flexible insulated sheath, each roller rod channel and each spring configured to arrest movement of each insulated sheath away from the colpotomy ring.
